(19) United States
(12) Patent Application Publication
(10) Pub. No.: US 2023/0144552 A1
(43) Pub. Date: May 11, 2023

(54) SALVAGE DEVICE AND METHOD FOR LOCALIZING AND REMOVING A BREAST TISSUE MARKER CLIP AT SURGERY AFTER A FAILED SPECIMEN RADIOGRAM

(71) Applicant: THE REGENTS OF THE UNIVERSITY OF CALIFORNIA, Oakland, CA (US)

(72) Inventors: Gregory Michael Senofsky, Pacific Palisades, CA (US); Nicholas Marlon Senofsky, Pacific Palisades, CA (US); Sloan Raven Senofsky, Pacific Palisades, CA (US)

(21) Appl. No.: 17/906,867

(22) PCT Filed: Mar. 24, 2021
(86) PCT No.: PCT/US21/23823

§ 371 Date: Sep. 21, 2022

Related U.S. Application Data
(60) Provisional application No. 63/000,348, filed on Mar. 26, 2020.

(51) Int. Cl. A61B 90/00; A61B 17/34

(52) U.S. Cl. CPC A61B 90/39; A61B 17/3468; A61B 2090/3908; A61B 2090/3966; A61B 2017/0092

(57) ABSTRACT

A salvage device for localizing a tissue marker clip includes an elongate handle having a metal ball connected to its distal end, and a probe connected to a distal end of the radiopaque fiducial element. A lumen extends through at least a portion of the elongate handle, the metal ball and the probe, terminating at a distal tip of the probe. A deployable tissue engagement member is housed at least partially within the lumen and connected to a proximal switch.

### CROSS-REFERENCE TO RELATED APPLICATIONS

This application claims priority to U.S. provisional application No. 63/000,348, filed on Mar. 26, 2020 and incorporated herein by reference in its entirety.

### BACKGROUND OF THE INVENTION

Breast imaging examinations can cause physicians to suspect that a patient has breast cancer. To verify, mammographers typically take a sample of tissue from the suspected tissue area of the breast via an image guided needle biopsy. The tissue sample is then examined by a pathologist to determine whether atypical or cancer cells are present. If atypical or cancer cells are present, the patient is then referred to a surgeon for planned surgical incision or lumpectomy based on the titanium clip that has been placed into the breast by the mammographer at the time of the needle biopsy in order to mark the area.

Various breast imaging biopsy techniques are available to mammographers. These include mammogram, ultrasound, and MRI image guidance, however all of these methods include the placement of a titanium clip into the breast tissue to mark a specific area of the biopsy in case the area needs to be surgically removed by a surgeon. All of these clips contain metal, including the titanium clips and the reflector clips. All of these clips are radiopaque.

So for example, a clip can be placed by a radiologist at the time of an image guided needle biopsy of the breast for a suspicious mammogram or ultrasound finding often times when no breast mass can be felt. When the image guided needle biopsy shows cancer or atypical cells, surgical excision of this area is warranted. Just prior to the surgery, a radiologist inserts a wire or tiny metal reflector into the breast to mark the clip for the surgeon. The clip must be removed with the tissue at the time of the surgery to assure that the correct breast tissue has been removed. This is confirmed by a specimen radiogram (an x-ray of the removed breast tissue at the time of surgery to prove that the clip is contained within the removed tissue).

Data shows that 97% of the time the clips are successfully removed at the time of surgery in the targeted breast tissue, but 3% of the time they are not. When the clip is not present in the breast tissue, this is referred to as a failed specimen radiogram. The suction canister which contains blood and irrigation fluid suctioned from the wound can be x rayed and sometimes the clip is in this fluid. Unfortunately, when the clip is not in the suction canister either, there currently exists no reliable alternative method of targeting the tiny clip still left in the breast after a failed specimen radiogram when the patient is still on the operating table. Surgeons are relegated to removing additional random breast tissue in the area in hopes of finding the clip, sometimes deforming the breast or telling the patient that they need a second surgery. The clips are usually too small to see by intraoperative ultrasound.

Thus, what is needed in the art is a salvage device and system for localizing and removing tissue marker clips after a failed specimen radiogram.

### SUMMARY OF THE INVENTION

A salvage device for localizing a tissue marker clip includes an elongate handle having a radiopaque fiducial element connected to its distal end, and a probe connected to a distal end of the radiopaque fiducial element; a lumen extending through at least a portion of the elongate handle, the radiopaque fiducial element and the probe, terminating at a distal tip of the probe; and a deployable tissue engagement member housed at least partially within the lumen and connected to a proximal switch. The salvage device is designed to be used on one patient only, and then discarded. In one embodiment, the elongate handle is radiolucent. In one embodiment, the elongate handle comprises plastic. In one embodiment, the radiopaque fiducial element comprises metal. In one embodiment, the radiopaque fiducial element is a metal ball. In one embodiment, the probe comprises a curved portion. In one embodiment, the deployable tissue engagement member comprises a plurality of tissue engagement members. In one embodiment, the deployable tissue engagement member comprises a plurality of barbs. In one embodiment, the deployable tissue engagement member comprises a plurality of members configured to assume a curved shape in a deployed state and a straight shape when housed within the lumen. In one embodiment, the deployable tissue engagement member comprises a shape memory material. In one embodiment, the shape memory material is a nitinol metal alloy. In one embodiment, the proximal switch slides distally to deploy the tissue engagement member and proximally to retract the tissue engagement member. In one embodiment, a kit includes the salvage device and a radiopaque ring. In one embodiment, a method for retrieving a tissue marker clip includes the steps of providing the kit; positioning a radiopaque ring around an incision site; imaging the ring and a target tissue site; advancing a salvage device into the target tissue site so that the radiopaque element is adjacent to the imaged clip; deploying tissue engagement elements; and removing the clip based on the location of a distal portion of the salvage device.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a side view of a salvage localization device (showing a partial cutaway) and radiopaque ring system according to one embodiment.

FIG. 2A is a side view of a salvage localization device (including a partial cutaway of the metal ball) with barbs in a retracted position according to one embodiment; and FIG. 2B is a side view of a salvage localization device with barbs in a deployed position according to one embodiment.

FIG. 3A is a diagram of a breast and incision site with a radiopaque ring placed around the incision site according to one embodiment; FIG. 3B shows a salvage device advanced to the target tissue site; and FIG. 3C shows the salvage device at the target tissue site with barbs deployed.

FIG. 4 is a flow chart of a method for retrieving a tissue marker clip according to one embodiment.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems and methods of localizing and removing a tissue marker clip. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a salvage device for localizing a tissue marker clip and a method for localizing and removing the clip.

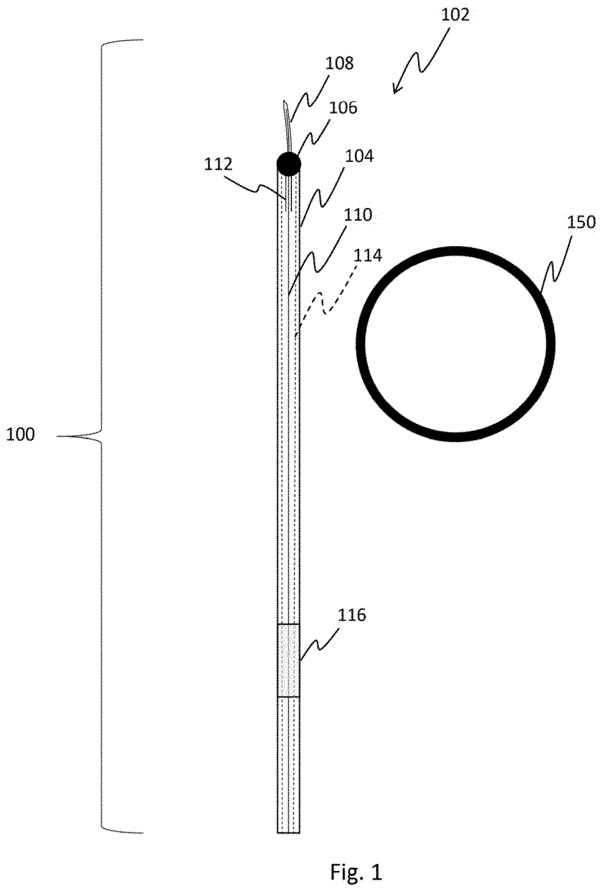

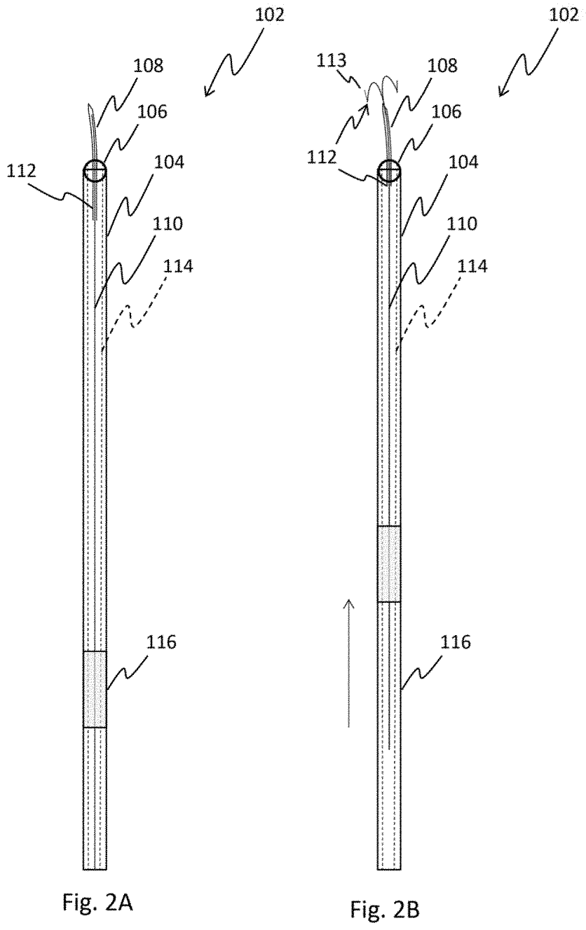

Embodiments of the device and method described herein are in certain embodiments utilized in concert with intraoperative fluoroscopy and performed as a salvage maneuver for a surgeon who has just performed an excisional breast biopsy or lumpectomy but has a failed specimen radiogram (i.e. the clip is not present in the lumpectomy specimen). With reference now to FIG. 1, a salvage device 102 and radiopaque ring 150 kit 100 shown according to one embodiment. The salvage device 102 for localizing a tissue marker clip includes an elongate handle 104 having a radiopaque fiducial element 106 connected to its distal end. A probe 108 is connected to a distal end of the salvage device 102. The elongate handle 104, radiopaque element 106 and probe 108 share a common lumen 114 extending therethrough and terminating at a distal tip of the probe 108. With additional reference now to FIGS. 2A and 2B, one or more deployable tissue engagement members 112 are housed at least partially within the lumen 114 and connected to a proximal switch 116 by an extension 110 to the members 112. The elongate handle 104 is radiolucent and in certain embodiments made of plastic. The radiopaque fiducial element 112 in certain embodiments is made of metal and has a generally rounded shape. Preferably, the probe 108 has a generally curved shape. The deployable tissue engagement member 112 can include multiple tissue engagement members with barbs 113 at the end. The deployable tissue engagement members 112 can be configured to assume a curved shape in a deployed state and a straight shape when housed within the lumen 114. This can be accomplished by utilizing for example a shape memory material such as a nitinol metal alloy. The proximal switch 116 slides distally to deploy the tissue engagement members 112 and proximally to retract the tissue engagement members 112.

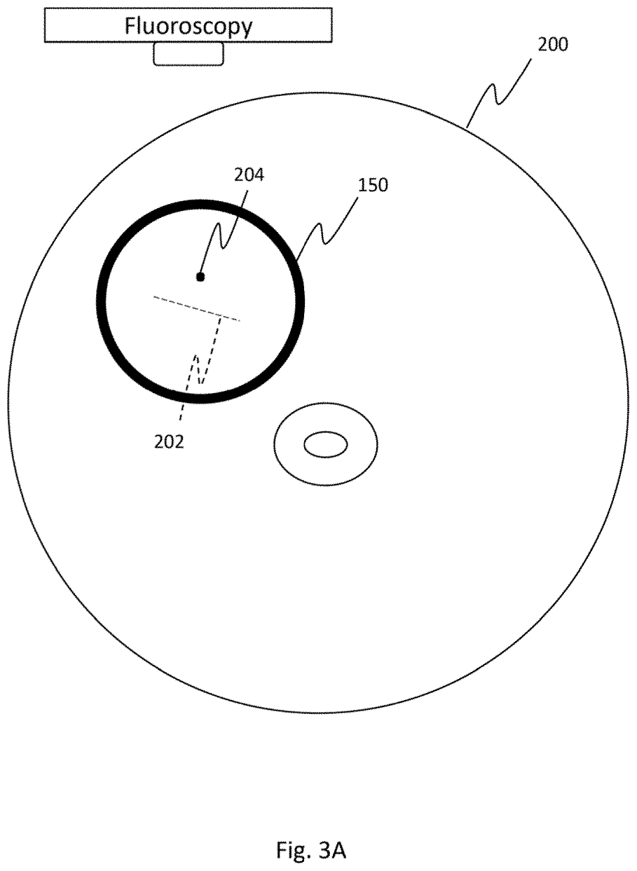

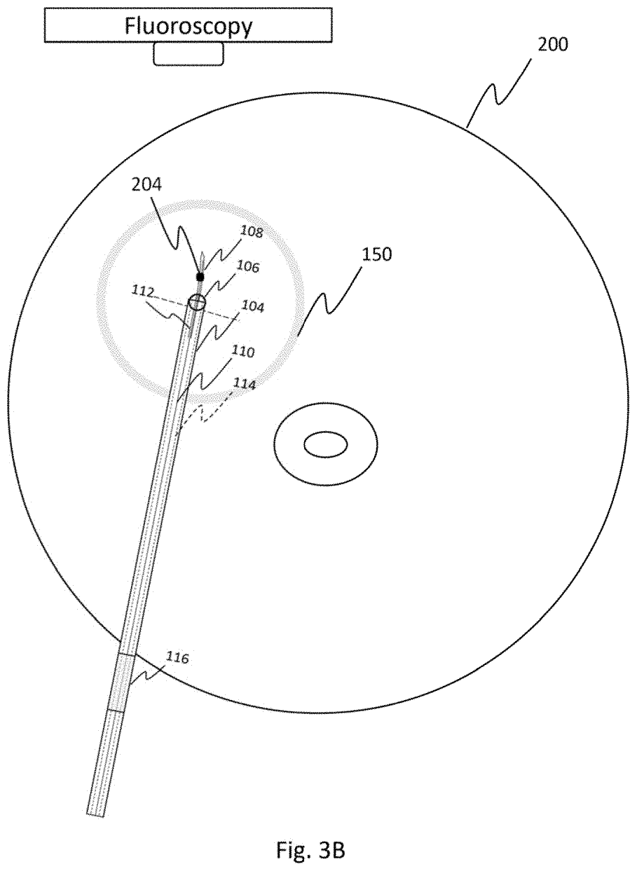

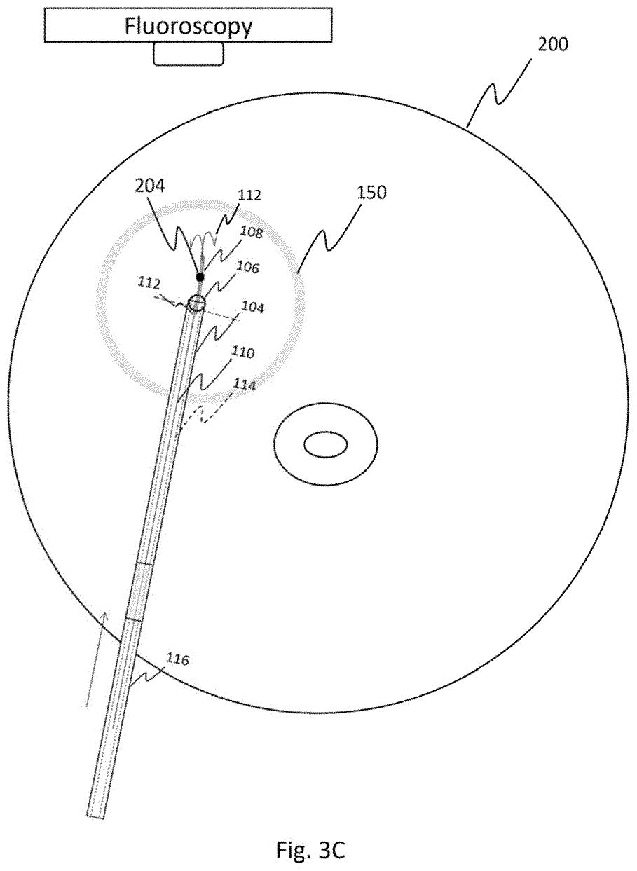

With reference now to FIGS. 3A-3C, a method for using the salvage device and retrieving a tissue marker clip is now described according to one embodiment. Two instruments, a salvage device 102 and radiopaque ring 150 which are sterilized and prepackaged (e.g. the kit of FIG. 1) are opened and handed to the surgeon after a negative specimen radiogram. The fluoroscopy machine which can see a tiny piece of metal is brought into the operating room and all personnel put on lead aprons and neck coverings and rescrub. The fluoroscopy machine is sterility draped and the radiopaque ring 150, which can be for example a 5 inch metal round ring ⅛ inch thick is positioned over and around the open incision 202 in the breast 200. The fluoroscopy camera is positioned directly over the ring 150 and images are taken until the fluoroscopy image as seen on the display screen shows the entire ring 150. A double magnification image is performed and if the clip 204 is still present in the breast 200, it can be seen with this method. If no clip 204 is seen, then the clip 204 is no longer in the breast 200 and the surgeon can be secure in terminating the surgical procedure.

If the clip 204 is seen the surgeon takes the salvage device, which can for example consist of an 8 inch long hollow clear plastic handle 104 connected to a 3 mm metal ball 106 with a hollow lumen 114 that connects to and terminates into a hollow ½ inch long slightly curved thin probe 108. At the other end of the 8 inch handle 104 is a switch 116 where the handle 104 flattens that can push two thin prongs 112 out through the tip of the probe 108 such that as they exit the tip of the probe 108 and they curve back so as to fix the probe 108 in the breast tissue like two hooks. The reason for the probe being slightly curved is to assist in spatial orientation. One side of the flattened handle can include colors or markers to assist with spatial orientation during repositioning of the probe in the breast tissue.

The surgeon holds the handle 104 and places the probe end into the wound and inserts the probe 108 into the edge of the lumpectomy cavity in the breast 200. Fluoroscopy images are taken as the probe 108 is repositioned until the metal ball 106 is closest to or adjacent to the clip 204. The fluoroscope can be tilted so as to obtain these imaged in orthogonal views to assure three dimensional orientation and accuracy. Once the probe 108 is in correct position the prongs 112 can be deployed to lock the probe 108 in position. The surgeon can then excise the tissue around the probe 108 and prongs 112. Repeat fluoroscopy will likely show that the clip has been removed and the breast tissue can be removed from the probe outside the body by withdrawing the prongs and submitted for specimen radiogram by the radiologist. If the clip has not been retrieved the process can be repeated until it has. The surgeon can then close the incision and send all oriented specimens for pathology. This process will complete the surgery with an effective and positive result. No random tissue removal is necessary in an attempt to retrieve the clip utilizing this system.

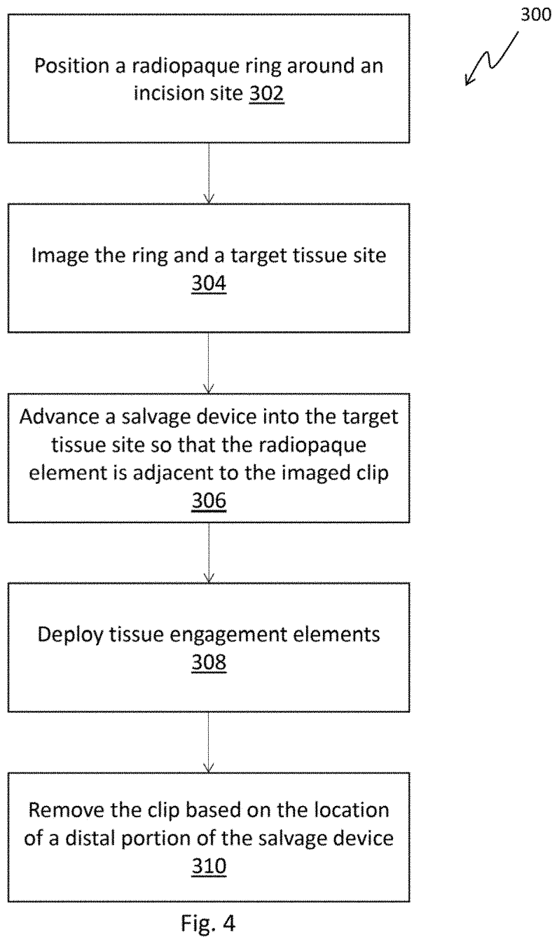

Thus, according to one embodiment and with reference now to FIG. 4, a method 300 for retrieving a tissue marker clip includes the steps of positioning a radiopaque ring around an incision site 302, imaging the ring and a target tissue site 304, advancing a salvage device into the target tissue site so that the radiopaque element is adjacent to the imaged clip 306, deploying tissue engagement elements 308, and removing the clip based on the location of a distal portion of the salvage device 310.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A salvage device for localizing a tissue marker clip comprising:
an elongate handle having a radiopaque fiducial element connected to its distal end, and a probe connected to a distal end of the radiopaque fiducial element;
a lumen extending through at least a portion of the elongate handle, the radiopaque fiducial element and the probe, terminating at a distal tip of the probe; and
a deployable tissue engagement member housed at least partially within the lumen and connected to a proximal switch.

2. The salvage device of claim 1, wherein the elongate handle is radiolucent.

3. The salvage device of claim 1, wherein the elongate handle comprises plastic.

4. The salvage device of claim 1, wherein the radiopaque fiducial element comprises metal.

5. The salvage device of claim 1, wherein the radiopaque fiducial element is a metal ball.

6. The salvage device of claim 1, wherein the probe comprises a curved portion.

7. The salvage device of claim 1, wherein the deployable tissue engagement member comprises a plurality of tissue engagement members.

8. The salvage device of claim 1, wherein the deployable tissue engagement member comprises a plurality of barbs.

9. The salvage device of claim 1, wherein the deployable tissue engagement member comprises a plurality of members configured to assume a curved shape in a deployed state and a straight shape when housed within the lumen.

10. The salvage device of claim 1, wherein the deployable tissue engagement member comprises a shape memory material.

11. The salvage device of claim 10, wherein the shape memory material is a nitinol metal alloy.

12. The salvage device of claim 1, wherein the proximal switch slides distally to deploy the tissue engagement member and proximally to retract the tissue engagement member.

13. A kit comprising the salvage device of claim 1 and a radiopaque ring.

14. A method for retrieving a tissue marker clip comprising:
providing the kit of claim 13;
positioning a radiopaque ring around an incision site;
imaging the ring and a target tissue site;
advancing a salvage device into the target tissue site so that the radiopaque element is adjacent to the imaged clip;
deploying tissue engagement elements; and
removing the clip based on the location of a distal portion of the salvage device.
